(19) United States
(12) Patent Application Publication
(10) Pub. No.: US 2020/0261259 A1
(43) Pub. Date: Aug. 20, 2020

(54) ELECTROTHERAPY INSOLE APPARATUS, SYSTEM AND METHOD

(71) Applicant: Quanqin Dai, Diamond Bar, CA (US)

(72) Inventor: Quanqin Dai, Diamond Bar, CA (US)

(21) Appl. No.: 16/278,841

(22) Filed: Feb. 19, 2019

(51) Int. Cl. A61F 7/00; A61N 1/04; A61N 1/36

(52) U.S. Cl. CPC A61F 7/007; A61N 1/0408; A61F 2007/0078; A61N 1/36021; A61F 2007/0045; A61N 1/0484

(57) ABSTRACT

An electrotherapy insole apparatus comprises an electrotherapy device and an insole. The electrotherapy device provides the electrotherapy stimulation and/or heat to the insole, and the insole contacts the foot of a user for the electrotherapy. The electrotherapy device attaches to and detaches from the insole. This allows the user to wash the insole if necessary. Also, this allows the user to replace the insole if necessary, providing an affordable replacement of insole and a long use of the electrotherapy device. The integrated circuit board inside the electrotherapy device is powered by the rechargeable battery or similar power source.

### FIELD OF THE INVENTION

The present invention relates to an electrotherapy insole apparatus, and more particularly to an electrotherapy insole apparatus, system and method capable of maintaining electrotherapy for pain relief and blood circulation increase.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

In the modern age of rapid economic development, people are busy with working or studying for a huge number of hours each week, which increases their daily psychological and physical pressure. Meanwhile, people also pay more and more attention to health, so that the needs of the electrotherapy devices for feet and other body areas are increased. This is because people may be standing or walking or driving for long hours in their daily commutes, or for their jobs, resulting in pain in their feet and other areas of their bodies. People are sitting more for their jobs during the day, which may result in weight gain, which then puts additional pressure on their feet. Furthermore, with less free time people have less of a chance to exercise, and so their feet and other parts of their body may be out of shape and more susceptible to pain.

Generally, the conventional electrotherapy footwear comprises a control unit, and a pair of conductive electrode pads which are electrically connected to the control unit through wires to provide electrical stimulation to user's feet.

The Chinese patent CN204908139U discloses a massage therapy shoe, but the cost of making such a footwear is high. In addition, it is impossible to make one size and one style to fit all. Therefore, it is not practical for the widespread use and there remains a need for an improved or new electrotherapy device for feet to overcome the problems presented above.

In some embodiments, the numbers expressing quantities of ingredients, properties Such as concentration, reaction conditions, and so forth, used to describe and claim certain embodiments of the invention are to be understood as being modified in some instances by the term “about.”

Accordingly, in some embodiments, the numerical parameters set forth in the written description and attached claims are approximations that can vary depending upon the desired properties sought to be obtained by a particular embodiment.

In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable.

The numerical values presented in some embodiments of the invention may contain certain errors necessarily resulting from the standard deviation found in their respective testing measurements.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

As used in the description herein and throughout the claims that follow, the meaning of “a,” “an” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “Such as’) provided with respect to certain embodiments herein is intended merely to better illuminate the invention and does not pose a limitation on the scope of the invention otherwise claimed.

No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

### SUMMARY OF THE INVENTION

The present invention provides an electrotherapy insole apparatus which comprises an electrotherapy device and an insole. The electrotherapy device is used to provide the electrotherapy to the insole, and the insole includes at least the first conductive area and the second conductive area. The integrated circuit board inside the electrotherapy device is powered by the rechargeable battery or similar power source.

In one embodiment, the electrotherapy device is attached onto the bottom of the insole.

In another embodiment, the insole does not include a heating layer.

In the third embodiment, the insole includes a heating layer.

This invention is advantageous because its use is more convenient and its replacement cost is much less than the conventional and existing electrotherapy footwears. The replacement cost is lower because the electrotherapy device can be removed from the bottom of the insole, allowing the insole to be replaced or washed, and thus maintain cleanliness. This prevents the need to replace the electrotherapy device itself, which would be much more costly. One of the reasons the electrotherapy device is removable is because it does not involve wires, and it is not permanently attached to the bottom of the insole. Also, the size of the insole could be trimmed to fit different sizes of foot and shoes, making it easy for widespread adoption by people with a variety of different shoe sizes.

### BRIEF DESCRIPTION OF THE DRAWINGS

FIG. 1 is the schematic view of one embodiment of the electrotherapy insole in the present invention.

FIG. 2 is the schematic view of another embodiment of the electrotherapy insole in the present invention.

FIG. 3 is the schematic view of the third embodiment of the electrotherapy insole in the present invention.

FIG. 4 is the view of the fourth embodiment of the present invention in use on a user's foot.

FIG. 5 is the schematic view of the remote control that is part of one embodiment of the present invention.

### DETAILED DESCRIPTION OF THE INVENTION

The detailed description set forth below is intended as a description of the presently exemplary device provided in accordance with aspects of the present invention and is not intended to represent the only forms in which the present invention may be prepared or utilized. It is to be understood, rather, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described can be used in the practice or testing of the invention, the exemplary methods, devices and materials are now described.

All publications mentioned are incorporated by reference for the purpose of describing and disclosing, for example, the designs and methodologies that are described in the publications that might be used in connection with the presently described invention. The publications listed or discussed above, below and throughout the text are provided solely for their disclosure prior to the filing date of the present application. Nothing herein is to be construed as an admission that the inventors are not entitled to antedate such disclosure by virtue of prior invention.

In order to further understand the goal, characteristics and effect of the present invention, a number of embodiments along with the drawings are illustrated as follows:

In the present invention of electrotherapy insole apparatus, the electrotherapy device can be detached to and attached from the insole, providing the electrotherapy to the insole. This allows the user to wash the insole if necessary. Also, this allows the user to replace the insole if necessary, providing an affordable replacement of insole and a long use of the electrotherapy device. The integrated circuit board inside the electrotherapy device is powered by the rechargeable battery or similar power source.

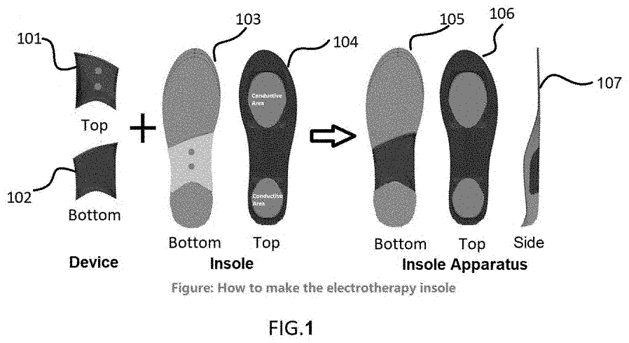

An embodiment of the invention is shown in FIG. 1. Here the two conductive contacts are shown on the top 101 of the electrotherapy device, while the bottom 102 of the electrotherapy device shows what that part of the electrotherapy insole apparatus will look like after being attached to the bottom 103 of the insole. The top of the insole 104 shows two lighter shaded areas that are the two conductive areas that will receive electrotherapy stimulation from the two conductive contacts on the top 101 of the electrotherapy device.

Then after a transformation, the bottom 105 of the electrotherapy insole apparatus shows the finished product of what the bottom 105 of the electrotherapy insole apparatus will look like after the electrotherapy device are placed on the bottom 103 of the insole. The top 106 of the electrotherapy insole apparatus again shows the same two lighter shaded areas that are the two conductive areas that will receive electrotherapy stimulation from the two conductive contacts on the top 101 of the electrotherapy device. The side view 107 shows the shape of the electrotherapy insole apparatus. The shape is that the insole is higher than the electrotherapy device at the back of the foot, and then after the electrotherapy device the insole slowly slopes downward until it is thinner towards the toe area of the insole.

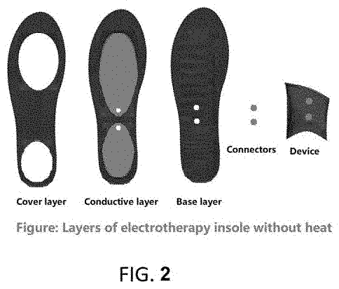

An additional embodiment of the invention is shown in FIG. 2, in which the structure of the electrotherapy insole apparatus is shown: A cover layer is the top of the insole, then the conductive layer is beneath the cover layer, and then the base layer is the bottom of the insole. The top of the electrotherapy device is attached to and detached from the base layer of the insole through the first and second conductive connectors to provide electrotherapy stimulation. The invention of FIG. 1 has such a structure described herein.

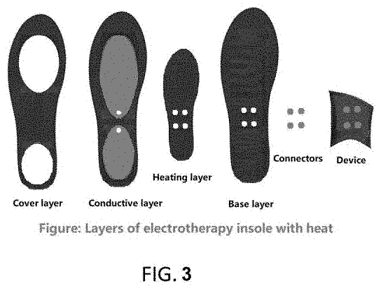

An additional embodiment of the invention is shown in FIG. 3, in which the invention of FIG. 1 is combined with a heating layer: A cover layer is the top of the insole, then the conductive layer is beneath the cover layer, then the heating layer is underneath the conductive layer, and then the base layer is the bottom of the insole. The top of the electrotherapy device is attached to and detached from the base layer of the insole to provide electrotherapy stimulation through the first and second conductive connectors and heat through the third and fourth conductive connectors.

The size of the electrotherapy insole is adjustable and trimmed to fit different sizes of shoes and feet.

Another embodiment of the present invention is a system of providing electrotherapy through an insole, comprising:
a bottom layer, which is the base of the insole and is used to protect an integrated circuit board;
the integrated circuit board, which provides the electrical stimulation to a conductive layer;
the conductive layer, which contacts the foot for the electrotherapy;
wherein the insole can be detached and reattached from the rest of the system multiple times;
wherein wireless remote operation can function utilizing a remote control that connects with the integrated circuit board through any of the following protocols: Bluetooth, Wi-Fi, radio frequency, and infrared;
wherein the integrated circuit board provides electrotherapy stimulation to at least a first conductive area and a second conductive area of the conductive layer through a first conductive contact and a second conductive contact; wherein the integrated circuit board is powered by a rechargeable battery; wherein a heating layer is placed underneath the conductive layer to provide heat;
wherein the rechargeable battery on the integrated circuit board provides power to the heating layer through a third conductive contact.

The wireless remote operation utilizes a remote that can communicate with the integrated circuit. The remote can adjust the intensity of the electrotherapy, as well as turning on and off the electrotherapy itself.

Another embodiment of the present invention is a system of providing electrical stimulation through an insole, comprising:
a bottom layer, which is the base of the insole and is used to protect an integrated circuit board;
the integrated circuit board, which provides the electrical stimulation to a conductive layer;
the conductive layer, which contacts the foot for the electrical stimulation; wherein the insole can be detached and reattached from the rest of the system multiple times;
wherein wireless remote operation can function utilizing a remote control that connects with the integrated circuit board through any of the following protocols: Bluetooth, Wi-Fi, radio frequency, and infrared;
wherein the integrated circuit board provides electrical stimulation to at least a first conductive area and a second conductive area of the conductive layer through a first conductive contact and a second conductive contact;
wherein the integrated circuit board is powered by a rechargeable battery; wherein a heating layer is placed underneath the conductive layer to provide heat;
wherein the rechargeable battery on the integrated circuit board provides power to the heating layer through a third conductive contact.

The wireless remote operation utilizes a remote that can communicate with the integrated circuit. The remote can adjust the intensity of the electrical stimulation, as well as turning on and off the electrical stimulation itself.

Another embodiment of the present invention is an apparatus of providing electrotherapy through an insole, comprising:

an electrotherapy device, which provides the electrotherapy stimulation and/or heat to the insole;
an insole, which contacts the foot of a user for the electrotherapy;
wherein the electrotherapy device attaches to and detaches from the insole;
wherein the integrated circuit board of the electrotherapy device provides electrotherapy stimulation to at least a first conductive area and a second conductive area of the conductive layer through the first and second conductive connectors;
wherein the integrated circuit board is powered by a rechargeable battery;
wherein the insole includes a cover layer as the top of the insole, then the conductive layer beneath the cover layer, then the heating layer is placed underneath the conductive layer to provide heat, and then the base layer as the bottom of the insole;
wherein a rechargeable battery on the integrated circuit board provides power to the heating layer through the third and fourth conductive connectors;
wherein wireless remote operation can function utilizing any of the following: Bluetooth, Wi-Fi, radio frequency, and infrared.

The wireless remote operation utilizes a remote that can communicate with the integrated circuit. The remote can adjust the intensity of the electrotherapy, as well as turning on and off the electrotherapy itself.

Another embodiment of the present invention is an apparatus of providing electrotherapy through an insole, comprising:

an electrotherapy device, which provides the electrotherapy stimulation and/or heat to the insole;
an insole, which contacts the foot of a user for the electrotherapy;
wherein the electrotherapy device attaches to and detaches from the insole;
wherein the integrated circuit board of the electrotherapy device provides electrotherapy stimulation to at least a first conductive area and a second conductive area of the conductive layer through the first and second conductive connectors;
wherein the integrated circuit board is powered by a rechargeable battery;
wherein the insole includes a cover layer as the top of the insole, then the conductive layer beneath the cover layer, and then the base layer as the bottom of the insole;
wherein a rechargeable battery on the integrated circuit board provides power to the heating layer through the third and fourth conductive connectors;
wherein wireless remote operation can function utilizing any of the following: Bluetooth, Wi-Fi, radio frequency, and infrared.

The wireless remote operation utilizes a remote that can communicate with the integrated circuit. The remote can adjust the intensity of the electrotherapy, as well as turning on and off the electrotherapy itself.

Another embodiment of the present invention is an apparatus of providing electrical stimulation through an insole, comprising:

an electrical stimulation device, which provides the electrical stimulation and/or heat to the insole;
an insole, which contacts the foot of a user for the electrical stimulation;
wherein the electrical stimulation device attaches to and detaches from the insole;
wherein the integrated circuit board of the electrical stimulation device provides electrical stimulation to at least a first conductive area and a second conductive area of the conductive layer through the first and second conductive connectors;
wherein the integrated circuit board is powered by a rechargeable battery;
wherein the insole includes a cover layer as the top of the insole, then the conductive layer beneath the cover layer, then the heating layer is placed underneath the conductive layer to provide heat, and then the base layer as the bottom of the insole;
wherein a rechargeable battery on the integrated circuit board provides power to the heating layer through the third and fourth conductive connectors;
wherein wireless remote operation can function utilizing any of the following: Bluetooth, Wi-Fi, radio frequency, and infrared.

The wireless remote operation utilizes a remote that can communicate with the integrated circuit. The remote can adjust the intensity of the electrical stimulation, as well as turning on and off the electrical stimulation itself.

Another embodiment of the present invention is an apparatus of providing electrical stimulation through an insole, comprising:

an electrical stimulation device, which provides the electrical stimulation and/or heat to the insole;
an insole, which contacts the foot of a user for the electrical stimulation;
wherein the electrical stimulation device attaches to and detaches from the insole;
wherein the integrated circuit board of the electrical stimulation device provides electrical stimulation to at least a first conductive area and a second conductive area of the conductive layer through the first and second conductive connectors;
wherein the integrated circuit board is powered by a rechargeable battery;
wherein the insole includes a cover layer as the top of the insole, then the conductive layer beneath the cover layer, and then the base layer as the bottom of the insole;
wherein a rechargeable battery on the integrated circuit board provides power to the heating layer through the third and fourth conductive connectors;
wherein wireless remote operation can function utilizing any of the following: Bluetooth, Wi-Fi, radio frequency, and infrared.

The wireless remote operation utilizes a remote that can communicate with the integrated circuit. The remote can adjust the intensity of the electrical stimulation, as well as turning on and off the electrical stimulation itself.

Another embodiment of the present invention is a system of providing electrotherapy through an insole, comprising:
a bottom layer, which is the base of the insole and is used to protect an integrated circuit board;
the integrated circuit board, which provides the electrical stimulation to a conductive layer;
the conductive layer, which contacts the foot for the electrotherapy;
wherein the insole can be detached and reattached from the rest of the system multiple times;
wherein wireless remote operation can function utilizing a remote control that connects with the integrated circuit board through any of the following protocols: Bluetooth, Wi-Fi, radio frequency, and infrared;
wherein the integrated circuit board provides electrotherapy stimulation to multiple conductive areas through multiple conductive contacts;
wherein the integrated circuit board is powered by a rechargeable battery;
wherein a heating layer is placed underneath the conductive layer to provide heat;
wherein the rechargeable battery on the integrated circuit board provides power to the heating layer through a third conductive contact.

The wireless remote operation utilizes a remote that can communicate with the integrated circuit. The remote can adjust the intensity of the electrotherapy, as well as turning on and off the electrotherapy itself.

Another embodiment of the present invention is a system of providing electrical stimulation through an insole, comprising:
a bottom layer, which is the base of the insole and is used to protect an integrated circuit board;
the integrated circuit board, which provides the electrical stimulation to a conductive layer;
the conductive layer, which contacts the foot for the electrical stimulation;
wherein the insole can be detached and reattached from the rest of the system multiple times;
wherein wireless remote operation can function utilizing a remote control that connects with the integrated circuit board through any of the following protocols: Bluetooth, Wi-Fi, radio frequency, and infrared;
wherein the integrated circuit board provides electrical stimulation to multiple conductive areas through multiple conductive contacts;
wherein the integrated circuit board is powered by a rechargeable battery;
wherein a heating layer is placed underneath the conductive layer to provide heat;
wherein the rechargeable battery on the integrated circuit board provides power to the heating layer through a third conductive contact.

The wireless remote operation utilizes a remote that can communicate with the integrated circuit. The remote can adjust the intensity of the electrical stimulation, as well as turning on and off the electrical stimulation itself.

Another embodiment of the present invention is an apparatus of providing electrotherapy through an insole, comprising:

an electrotherapy device, which provides the electrotherapy stimulation and/or heat to the insole;
an insole, which contacts the foot of a user for the electrotherapy;
wherein the electrotherapy device attaches to and detaches from the insole;
wherein the integrated circuit board of the electrotherapy device provides electrotherapy stimulation to multiple conductive areas through multiple conductive connectors;
wherein the integrated circuit board is powered by a rechargeable battery;
wherein the insole includes a cover layer as the top of the insole, then the conductive layer beneath the cover layer, then the heating layer is placed underneath the conductive layer to provide heat, and then the base layer as the bottom of the insole;
wherein a rechargeable battery on the integrated circuit board provides power to the heating layer through the third and fourth conductive connectors;
wherein wireless remote operation can function utilizing any of the following: Bluetooth, Wi-Fi, radio frequency, and infrared.

The wireless remote operation utilizes a remote that can communicate with the integrated circuit. The remote can adjust the intensity of the electrotherapy, as well as turning on and off the electrotherapy itself.

Another embodiment of the present invention is an apparatus of providing electrotherapy through an insole, comprising:

an electrotherapy device, which provides the electrotherapy stimulation and/or heat to the insole;
an insole, which contacts the foot of a user for the electrotherapy;
wherein the electrotherapy device attaches to and detaches from the insole;
wherein the integrated circuit board of the electrotherapy device provides electrotherapy stimulation to multiple conductive areas through multiple conductive connectors;
wherein the integrated circuit board is powered by a rechargeable battery;
wherein the insole includes a cover layer as the top of the insole, then the conductive layer beneath the cover layer, and then the base layer as the bottom of the insole;
wherein a rechargeable battery on the integrated circuit board provides power to the heating layer through the third and fourth conductive connectors;
wherein wireless remote operation can function utilizing any of the following: Bluetooth, Wi-Fi, radio frequency, and infrared.

The wireless remote operation utilizes a remote that can communicate with the integrated circuit. The remote can adjust the intensity of the electrotherapy, as well as turning on and off the electrotherapy itself.

Another embodiment of the present invention is an apparatus of providing electrical stimulation through an insole, comprising:

an electrical stimulation device, which provides the electrical stimulation and/or heat to the insole;
an insole, which contacts the foot of a user for the electrical stimulation;
wherein the electrical stimulation device attaches to and detaches from the insole;
wherein the integrated circuit board of the electrical stimulation device provides electrical stimulation to multiple conductive areas through multiple conductive connectors;
wherein the integrated circuit board is powered by a rechargeable battery;
wherein the insole includes a cover layer as the top of the insole, then the conductive layer beneath the cover layer, then the heating layer is placed underneath the conductive layer to provide heat, and then the base layer as the bottom of the insole;
wherein a rechargeable battery on the integrated circuit board provides power to the heating layer through the third and fourth conductive connectors;
wherein wireless remote operation can function utilizing any of the following: Bluetooth, Wi-Fi, radio frequency, and infrared.

The wireless remote operation utilizes a remote that can communicate with the integrated circuit. The remote can adjust the intensity of the electrical stimulation, as well as turning on and off the electrical stimulation itself.

Another embodiment of the present invention is an apparatus of providing electrical stimulation through an insole, comprising:

an electrical stimulation device, which provides the electrical stimulation and/or heat to the insole;
an insole, which contacts the foot of a user for the electrical stimulation;
wherein the electrical stimulation device attaches to and detaches from the insole;
wherein the integrated circuit board of the electrical stimulation device provides electrical stimulation to multiple conductive areas through multiple conductive connectors;
wherein the integrated circuit board is powered by a rechargeable battery;
wherein the insole includes a cover layer as the top of the insole, then the conductive layer beneath the cover layer, and then the base layer as the bottom of the insole;
wherein a rechargeable battery on the integrated circuit board provides power to the heating layer through the third and fourth conductive connectors;
wherein wireless remote operation can function utilizing any of the following: Bluetooth, Wi-Fi, radio frequency, and infrared.

The wireless remote operation utilizes a remote or phone App that can communicate with the integrated circuit. The remote or phone App can adjust the intensity of the electrical stimulation, as well as turning on and off the electrical stimulation itself.

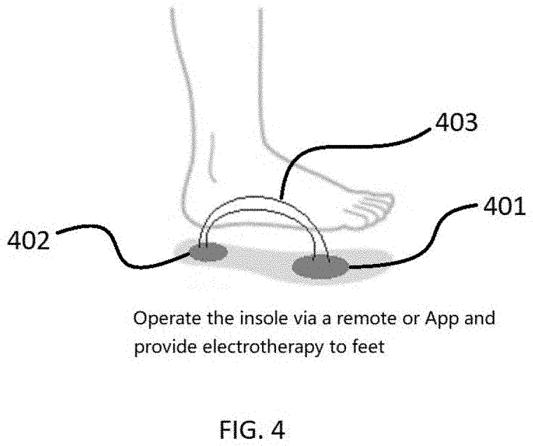

FIG. 4 displays a foot experiencing electrotherapy through one embodiment of the present invention. The foot is on top of a first conductive area 401 and a second conductive area 402, which are providing electrotherapy to the foot in the form of arc-like patterns 403. These arc-like patterns 403 radiate throughout the foot, and can help relieve pain and increase blood circulation. In another embodiment of the present invention the arc-like patters 403 also radiate heat through the foot. The combination of heat and electrotherapy provides additional pain relief in the foot, more so than electrotherapy alone.

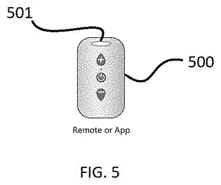

FIG. 5 displays a remote 500 through which the user can control the electrotherapy being sent through the present invention through the foot of the user. The power button in the center turns on the electrotherapy stimulation and/or heat. The plus button increases the power sent through the conductive areas of the present invention, thereby increasing the electrical stimulation and providing increased electrotherapy. The minus button decreases the power sent through the conductive areas of the present invention, thereby decreasing the electrical stimulation and providing reduced electrotherapy. The optional key ring 501 provides convenience for the user to carry the remote.

Having described the invention by the description and illustrations above, it should be understood that these are exemplary of the invention and are not to be considered as limiting. Accordingly, the invention is not to be considered as limited by the foregoing description, but includes any equivalents.

What is claimed is:

1. An apparatus of providing electrotherapy through an insole, comprising:
an electrotherapy device, which provides the electrotherapy stimulation and/or heat to the insole;
an insole, which contacts the foot of a user for the electrotherapy;
the electrotherapy device attaches to and detaches from the insole.

2. The apparatus of claim 1, further comprising:
wherein the integrated circuit board of the electrotherapy device provides electrotherapy stimulation to at least a first conductive area and a second conductive area of the conductive layer through the first and second conductive connectors.

3. The apparatus of claim 1, further comprising:
wherein the integrated circuit board is powered by a rechargeable battery.

4. The apparatus of claim 1, further comprising:
wherein wireless remote operation can function utilizing any of the following:
Bluetooth, Wi-Fi, radio frequency, and infrared.

5. The apparatus of claim 1, further comprising:
wherein the insole includes a cover layer as the top of the insole, then the conductive layer beneath the cover layer, and then the base layer as the bottom of the insole.

6. The apparatus of claim 1, further comprising:
wherein the insole includes a cover layer as the top of the insole, then the conductive layer beneath the cover layer, then a heating layer is placed underneath the conductive layer to provide heat, and then the base layer as the bottom of the insole.

7. The apparatus of claim 5, further comprising:
wherein a rechargeable battery on the integrated circuit board provides power to the heating layer through the third and fourth conductive connectors.

8. A system of providing electrotherapy through an insole, comprising:
an electrotherapy device, which provides the electrotherapy stimulation and/or heat to the insole;
an insole, which contacts the foot of a user for the electrotherapy;
the electrotherapy device attaches to and detaches from the insole;
wherein wireless remote operation can function utilizing any of the following:
Bluetooth, Wi-Fi, radio frequency, and infrared.

9. The system of claim 8, further comprising:
wherein the integrated circuit board of the electrotherapy device provides electrotherapy stimulation to at least a first conductive area and a second conductive area of the conductive layer through the first and second conductive connectors.

10. The system of claim 8, further comprising:
wherein the integrated circuit board is powered by a rechargeable battery.

11. The system of claim 8, further comprising:
wherein the insole includes a cover layer as the top of the insole, then the conductive layer beneath the cover layer, and then the base layer as the bottom of the insole.

12. The system of claim 8, further comprising:
wherein the insole includes a cover layer as the top of the insole, then the conductive layer beneath the cover layer, then a heating layer is placed underneath the conductive layer to provide heat, and then the base layer as the bottom of the insole.

13. The system of claim 12, further comprising:
wherein a rechargeable battery on the integrated circuit board provides power to the heating layer through the third and fourth conductive connectors.

14. A method of providing electrotherapy through an insole, comprising:
an electrotherapy device, which provides the electrotherapy stimulation and/or heat to the insole;
an insole, which contacts the foot of a user for the electrotherapy;
the electrotherapy device attaches to and detaches from the insole;
wherein wireless remote operation can function utilizing any of the following:
Bluetooth, Wi-Fi, radio frequency, and infrared.

15. The method of claim 14, further comprising:
wherein the integrated circuit board of the electrotherapy device provides electrotherapy stimulation to at least a first conductive area and a second conductive area of the conductive layer through the first and second conductive connectors.

16. The method of claim 14, further comprising:
wherein the integrated circuit board is powered by a rechargeable battery.

17. The method of claim 14, further comprising:
wherein the insole includes a cover layer as the top of the insole, then the conductive layer beneath the cover layer, and then the base layer as the bottom of the insole.

18. The method of claim 14, further comprising:
wherein the insole includes a cover layer as the top of the insole, then the conductive layer beneath the cover layer, then a heating layer is placed underneath the conductive layer to provide heat, and then the base layer as the bottom of the insole.

19. The method of claim 18, further comprising:
wherein a rechargeable battery on the integrated circuit board provides power to the heating layer through the third and fourth conductive connectors.

20. The method of claim 14, further comprising:
wherein the integrated circuit board of the electrotherapy device provides electrotherapy stimulation to at least a first conductive area and a second conductive area of the conductive layer through the first and second conductive connectors;
wherein the integrated circuit board is powered by a rechargeable battery;
wherein the insole includes a cover layer as the top of the insole, then the conductive layer beneath the cover layer, then a heating layer is placed underneath the conductive layer to provide heat, and then the base layer as the bottom of the insole;
wherein a rechargeable battery on the integrated circuit board provides power to the heating layer through the third and fourth conductive connectors.
